(19) United States
(12) Patent Application Publication
(10) Pub. No.: US 2025/0250610 A1
(43) Pub. Date: Aug. 7, 2025

(54) METHODS AND SYSTEMS FOR THERAPEUTIC AGENT ANALYSIS

(71) Applicant: ReadCoor, LLC, Pleasanton, CA (US)

(72) Inventors: Evan Daugharthy, Cambridge, MA (US); Richard Terry, Carlisle, MA (US)

(21) Appl. No.: 18/955,731

(22) Filed: Nov. 21, 2024

Related U.S. Application Data
(63) Continuation of application No. 17/190,258, filed on Mar. 2, 2021, now abandoned.
Application No. 17/190,258 is a continuation of application No. PCT/US2019/049542, filed on Sep. 4, 2019.
(60) Provisional application No. 62/727,295, filed on Sep. 5, 2018.

(51) Int. Cl. C12Q 1/6809

(52) U.S. Cl. CPC C12Q 1/6809

(57) ABSTRACT

The present disclosure provides methods and systems for analyzing agents (e.g., therapeutic agents) in a biological sample having a three-dimensional matrix.

### CROSS-REFERENCE

This application is a continuation of U.S. patent application Ser. No. 17/190,258 filed Mar. 2, 2021, which is a continuation of International Application No. PCT/US2019/049542, filed Sep. 4, 2019, which claims priority to U.S. Provisional Patent Application No. 62/727,295, filed Sep. 5, 2018, each of which is entirely incorporated herein by reference.

### BACKGROUND

Therapeutic agents such as small interfering RNAs (siRNA) and microRNAs (miRNA) are transforming the therapeutic landscape from cancer to neurologic disorders. However, few molecules have reached FDA approval. Challenges in delivery verification and off target knockdowns represent a significant barrier to clinical utility. Bulk RNA sequencing can determine global transcriptional changes; however, small effects are often lost in the noise. In addition, due to homogenizing the samples, bulk RNA sequencing cannot localize effects to specific tissues, cell-types, or individual cells. Fluorescent in situ hybridization targeting ribonucleic acid molecules (RNA-FISH) can localize transcriptional changes to specific cells, however, its low multiplexity limits the number of targets observed. Finally, RNA-FISH may require long probes (250-1,500 bases), thus it cannot directly detect and localize these small molecules, 20-50 bases.

### SUMMARY

The present disclosure provides methods and systems for therapeutic agent detection in a biological sample having a three-dimensional matrix.

According to an aspect, provided herein is a method for identifying an anti-sense nucleic acid molecule and a target molecule, comprising: (a) providing a biological sample having or suspected of having the target molecule and the anti-sense nucleic acid molecule, wherein the biological sample comprises a three-dimensional (3D) matrix; (b) using detection probes separate from the anti-sense nucleic acid molecule and the target molecule to detect a first set of signals and a second set of signals from the biological sample, wherein the first set of signals identifies a relative position of the anti-sense nucleic acid molecule in the biological sample, and wherein the second set of signals identifies an increase or a decrease in a level of the target molecule relative to a reference; and (c) using the first set of signals and the second set of signals to provide an output indicative of a position of each of the anti-sense nucleic acid molecule and the target molecule in the biological sample.

In some embodiments, the method further comprises, prior to (a), providing the biological sample, contacting the biological sample with a solution having the anti-sense nucleic acid molecule, and generating the 3D matrix subsequent to contacting the biological sample with the solution. In some embodiments, the target molecule is a target nucleic acid molecule.

In some embodiments, (b) comprises using the detection probes to identify a target sequence of the target nucleic acid molecule, thereby providing the second set of signals. In some embodiments, (b) comprises using the detection probes to sequence the target nucleic acid molecule, thereby identifying the target sequence. In some embodiments, (b) comprises using the detection probes to identify an anti-sense sequence of the anti-sense nucleic acid molecule, thereby providing the first set of signals. In some embodiments, (b) comprises using the detection probes to sequence the anti-sense nucleic acid molecule, thereby identifying the anti-sense sequence.

In some embodiments, the target molecule is a target polypeptide or protein. In some embodiments, the biological sample is a cell, a cell derivative, or a tissue. In some embodiments, the anti-sense nucleic acid molecule is a therapeutic agent. In some embodiments, the output is an image or video. In some embodiments, the anti-sense nucleic acid molecule is a ribonucleic acid, a phosphorothioate deoxyribonucleic acid, a locked nucleic acid, 2′-O-methocy-ethyl ribonucleic acid, 2′-O-methyl ribonucleic acid, or a 2′-fluoro deoxyribonucleic acid. In some embodiments, the anti-sense nucleic acid molecule is from 15 to 60 nucleotides in length.

According to another aspect, provided herein is a method for identifying an anti-sense ribonucleic (RNA) molecule co-localized with a target nucleic RNA molecule, comprising: (a) providing a biological sample having or suspected of having the target RNA molecule and the anti-sense RNA molecule of the target RNA molecule, wherein the biological sample comprises a three-dimensional (3D) matrix; (b) detecting a first set of signals and a second set of signals from the biological sample, wherein the first set of signals identifies a relative position of the anti-sense RNA molecule in the biological sample, and wherein the second set of signals identifies an increase or a decrease in a level of the target RNA molecule relative to a reference; and (c) using the first set of signals and the second set of signals to provide an output indicative of the anti-sense RNA molecule co-localized with the target RNA molecule in the biological sample.

In some embodiments, the method further comprises, prior to (a), providing the biological sample, contacting the biological sample with a solution having the anti-sense RNA molecule, and generating the 3D matrix subsequent to contacting the biological sample with the solution. In some embodiments, the biological sample is a cell, a cell derivative, or a tissue. In some embodiments, (b) comprises using detection probes to identify an anti-sense sequence of the anti-sense RNA molecule, thereby providing the first set of signals. In some embodiments, (b) comprises using the detection probes to sequence the anti-sense RNA molecule, thereby identifying the anti-sense sequence. In some embodiments, (b) comprises using detection probes to identify a target sequence of the target RNA molecule, thereby providing the second set of signals. In some embodiments, (b) comprises using the detection probes to sequence the target RNA molecule, thereby identifying the target sequence. In some embodiments, the anti-sense nucleic acid molecule is a therapeutic agent. In some embodiments, the output is an image or video. In some embodiments, the anti-sense RNA molecule is a modified anti-sense nucleic acid molecule. In some embodiments, the modified anti-sense nucleic acid molecule is a ribonucleic acid, a phosphorothioate deoxyribonucleic acid, a locked nucleic acid, 2′-O-methocy-ethyl ribonucleic acid, 2′-O-methyl ribonucleic acid, or a 2′-fluoro deoxyribonucleic acid. In some embodiments, the anti-sense RNA molecule is from 15 to 60 nucleotides in length.

According to another aspect, provided herein is a method for identifying two or more genetic aberrations of a target nucleic acid, comprising: (a) providing a biological sample having or suspected of having at least a first genetic aberration and a second genetic aberration of the target nucleic acid, wherein the biological sample comprises a three-dimensional (3D) matrix; (b) using detection probes to detect a first set of signals and a second set of signals from the biological sample, wherein the first set of signals identifies a first relative position of the first genetic aberration in the biological sample, and wherein the second set of signals identifies a second relative position of the second genetic aberration in the biological sample; and (c) using the first set of signals and the second set of signals to provide an output indicative of a position of each of the first genetic aberration and the second genetic aberration in the biological sample.

In some embodiments, the detection probes are targeted to a same sequence of the first genetic aberration and the second genetic aberration. In some embodiments, the first genetic aberration and the second genetic aberration have at least a single nucleotide difference. In some embodiments, the first genetic aberration comprises an additional sequence than the second genetic aberration. In some embodiments, the biological sample is a cell, a cell derivative, or a tissue.

According to another aspect, provided herein is a method for identifying two or more locations of an anti-sense nucleic acid molecule, comprising: (a) providing a biological sample having the anti-sense nucleic acid molecule, wherein the biological sample comprises a three-dimensional (3D) matrix; (b) using a detection probe to detect a set of signals of the anti-sense nucleic acid molecule within the biological sample; (c) detecting a first set of signals and a second set of signals from at least a first location and a second location within the biological sample, wherein the first location and the second location are within different sub-cellular compartments; and (d) using the set of signals of the anti-sense nucleic acid molecule and the first and the second set of signals of the first location and the second location to provide an output indicative of the anti-sense nucleic acid molecule co-localized with the first location and the second location.

In some embodiments, the anti-sense nucleic acid molecule is from 15 to 60 nucleotides in length. In some embodiments, the sub-cellular compartments comprise mitochondria, endoplasmic reticulum, P-bodies, Golgi apparatus, vesicle, endosome, lysosome, nucleus, microtubule, or a combination thereof. In some embodiments, the anti-sense nucleic acid molecule is a therapeutic agent. In some embodiments, the biological sample is a cell, a cell derivative, or a tissue.

According to another aspect, provided herein is a method for identifying co-localized molecules in a biological sample, comprising: (a) providing the biological sample having or suspected of having a target molecule and a an agent capable of binding or interacting with the target molecule, wherein the biological sample comprises a three-dimensional (3D) matrix; (b) using detection probes separate from the target molecule and the agent to detect a first set of signals and a second set of signals from the biological sample, wherein the first set of signals identifies a relative position of the target molecule in the biological sample, and wherein the second set of signals identifies a relative position of the agent in the biological sample; and (c) using the first set of signals and the second set of signals to provide an output indicative of the target molecule co-localized with the agent in the biological sample.

In some embodiments, the agent is a therapeutic agent. In some embodiments, the therapeutic agent is a polynucleotide, a polypeptide, or a small molecule.

According to another aspect, provided herein is a method for identifying an agent and a metabolite of the agent in a biological sample, comprising: (a) providing the biological sample having or suspected of having the agent and the metabolite of the agent, wherein the biological sample comprises a three-dimensional (3D) matrix; (b) using detection probes separate from the target molecule and the agent to detect a first set of signals and a second set of signals from the biological sample, wherein the first set of signals identifies a relative position of the agent in the biological sample, and wherein the second set of signals identifies a relative position of the metabolite in the biological sample; and (c) using the first set of signals and the second set of signals to provide an output indicative of a position of each of the agent and the metabolite.

In some embodiments, the biological sample is a cell, a cell derivative, or a tissue. In some embodiments, the agent is a therapeutic agent. In some embodiments, the therapeutic agent is a polynucleotide, a polypeptide, or a small molecule.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A depicts an example image of fluorescent in situ sequencing (FISSEQ) established culture models for brain cell types. The minimal targeted FISSEQ knock-down assay can be developed to test RNAi & anti-sense modalities across brain cell types.

FIG. 1B depicts an example image of sub-cellular compartment.

FIG. 1C depicts example genes scored based on (1) localization; and (2) variation in expression and generality across cell types.

FIG. 2A depicts an example image of using minimal targeted FISSEQ knock-down assay to screen physical-chemical and receptor-mediated delivery mechanisms of RNAi and anti-sense modalities in ICV tissue model.

FIG. 2B depicts example experimental data showing target mRNA level in response to therapeutic RNA dose.

FIG. 3A depicts a schematic of using FISSEQ probe for genetic aberration (e.g., gene variants) detection.

FIG. 3B depicts example experimental data showing two gene variants detected in FISSEQ assay.

FIG. 4A depicts a schematic of using FISSEQ probe for gene expression detection.

FIG. 4B depicts example experimental data of gene expression spatial distribution.

FIG. 5A depicts an example image of rough endoplasmic reticulum (ER) within a cell.

FIG. 5B depicts an example image of mitochondria distributed within a cell.

FIG. 5C depicts an example image of therapeutic RNA distributed within a cell.

FIG. 6A depicts in situ anti-sense oligonucleotide (ASO) and knockdown detection in mouse brain. The image shows detection of ASO on control ASO injected brain. The background signal shows probe specific for therapeutic ASO does not bind off target.

FIG. 6B depicts in situ ASO and knockdown detection in mouse brain. The images shows detection of ASO on therapeutic ASO injected brain. Signal at injection site and through cerebellum confirms therapeutic ASO detection and localization to expected brain areas.

FIG. 6C depicts in situ ASO and knockdown detection in mouse brain. The image shows MALAT 1, the target of the therapeutic ASO, is present in high abundance in the control.

FIG. 6D depicts in situ ASO and knockdown detection in mouse brain. The images shows MALAT1 is knocked down with the therapeutic ASO.

FIG. 6E depicts raw intensity quantified from images in FIG. 6A and FIG. 6B.

FIG. 6F depicts raw intensity quantified from images in FIG. 6C and FIG. 6D.

FIG. 7A depicts example gene expression profile in astrocyte (repeat 1) detected by FISSEQ.

FIG. 7B depicts example gene expression profile in astrocyte (repeat 2) detected by FISSEQ.

FIG. 7C depicts example gene expression profile in astrocyte (repeat 3) detected by FISSEQ.

FIG. 7D depicts example gene expression profile in astrocyte (repeat 4) detected by FISSEQ.

FIG. 7E depicts example gene expression profile in astrocyte (repeat 5) detected by bulk RNA sequencing. The data from repeats 1˜4 correlate with the bulk RNA sequencing data.

FIG. 8A depicts ASO knockdown and localization in astrocyte cell culture and dose response of H₂O, control ASO, and therapeutic ASO to knockdown MALAT1. The image shows detection of MALAT1 in cell culture treated with H₂O.

FIG. 8B depicts ASO knockdown and localization in astrocyte cell culture and dose response of H₂O, control ASO, and therapeutic ASO to knockdown MALAT1. The image shows detection of MALAT1 in cell culture treated with control ASO.

FIG. 8C depicts ASO knockdown and localization in astrocyte cell culture and dose response of H₂O, control ASO, and therapeutic ASO to knockdown MALAT1. The image shows detection of MALAT1 in cell culture treated with 0.5 μM therapeutic ASO.

FIG. 8D depicts ASO knockdown and localization in astrocyte cell culture and dose response of H₂O, control ASO, and therapeutic ASO to knockdown MALAT1. The image shows detection of MALAT1 in cell culture treated with 5.0 μM therapeutic ASO.

FIG. 8E depicts ASO knockdown and localization in astrocyte cell culture and dose response of H₂O, control ASO, and therapeutic ASO to knockdown MALAT1. The experimental data show intensity per area quantified from images in FIGS. 8A-D.

FIG. 9A depicts ASO knockdown in astrocyte cell culture correlated with qPCR: FISSEQ detected a ˜7-fold reduction in MALAT1 from therapeutic ASO 5 μM compared to control ASO. The image shows MALAT1 detection in cell culture treated with control ASO.

FIG. 9B depicts ASO knockdown in astrocyte cell culture correlated with qPCR: FISSEQ detected a ˜7-fold reduction in MALAT1 from therapeutic ASO 5 μM compared to control ASO. The image shows MALAT1 detection in cell culture treated with therapeutic ASO.

FIG. 9C depicts ASO knockdown in astrocyte cell culture correlated with qPCR: FISSEQ detected a ˜7-fold reduction in MALAT1 from therapeutic ASO 5 μM compared to control ASO. The qPCR data are shown as the bar graph. Two repeats were tested.

FIG. 10 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” or “the” includes plural references unless the context clearly dictates otherwise. For example, the term “a cell” includes a plurality of cells, including mixtures thereof.

The terms “amplifying” and “amplification,” as used herein, generally refer to generating one or more copies (or “amplified product” or “amplification product”) of a nucleic acid. The one or more copies may be generated by nucleic acid extension. Such extension may be a single round of extension or multiple rounds of extension. The amplified product may be generated by polymerase chain reaction (PCR).

The term “reverse transcription,” as used herein, generally refers to the generation of a deoxyribonucleic acid (DNA) molecule from a ribonucleic acid (RNA) molecule via the action of a reverse transcription enzyme (or reverse transcriptase).

The term “nucleic acid,” as used herein, generally refers to a nucleic acid molecule comprising a plurality of nucleotides or nucleotide analogs. A nucleic acid may be a polymeric form of nucleotides. A nucleic acid may comprise deoxyribonucleotides and/or ribonucleotides, or analogs thereof. A nucleic acid may be an oligonucleotide or a polynucleotide. Nucleic acids may have various three-dimensional structures and may perform various functions. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation, with a functional moiety for immobilization.

The term “subject,” as used herein, generally refers to an entity or a medium that has testable or detectable genetic information. A subject can be a person or an individual. A subject can be a vertebrate, such as, for example, a mammal. Non-limiting examples of mammals include murines, simians, and humans. A subject may be an animal, such as a farm animal. A subject may be a pet, such as dog, cat, mouse, rat, or bird. Other examples of subjects include food, plant, soil, and water. A subject may be displaying a disease. As an alternative, the subject may be asymptomatic.

Any suitable biological sample that comprises nucleic acid may be obtained from a subject. Any suitable biological sample that comprises nucleic acid may be used in the methods and systems described herein. A biological sample may be solid matter (e.g., biological tissue) or may be a fluid (e.g., a biological fluid). In general, a biological fluid can include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. A biological sample may be a cell-free sample. Such cell-free sample may include DNA and/or RNA.

The term “reactive group” or “functional moiety,” as used herein, generally refers to any moiety on a first reactant that is capable of reacting chemically with another functional moiety or reactive group on a second reactant to form a covalent or ionic linkage. “Reactive group” and “functional moiety” may be used interchangeably. For example, a reactive group of the monomer or polymer of the matrix-forming material can react chemically with a functional moiety (or another reactive group) on the substrate of interest or the target to form a covalent or ionic linkage. The substrate of interest or the target may then be immobilized to the matrix via the linkage formed by the reactive group and the functional moiety. Examples of suitable reactive groups or functional moieties include electrophiles or nucleophiles that can form a covalent linkage by reaction with a corresponding nucleophile or electrophile, respectively, on the substrate of interest. Non-limiting examples of suitable electrophilic reactive groups may include, for example, esters including activated esters (such as, for example, succinimidyl esters), amides, acrylamides, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups may include, for example, amines, anilines, thiols, alcohols, phenols, hyrazines, hydroxylamines, carboxylic acids, glycols, heterocycles, and the like.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” can mean within an order of magnitude, such as within 5-fold or within 2-fold of a value. Where particular values are described in the application and claims, unless otherwise stated the term “about” meaning within an acceptable error range for the particular value should be assumed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure provides methods and systems for fluorescent in situ Sequencing (FISSEQ). FISSEQ can be used to spatially localize transcriptional changes with therapeutic agents in tissue and cell culture. FISSEQ refers to a three-dimensional (3D) spatial panomic molecular detection technology that can deliver global transcriptome, genome, and proteome profiles within the tissue-microenvironment. FISSEQ results can include an image rendering of the sample, in addition to sequences tagged with their coordinates and any variants detected. Examples of these variants include, but are not limited to, single nucleotide polymorphisms (SNPs), deletions, and insertions. With the ability to measure genes and proteins at the ‘omic’ scale, FISSEQ can provide deeper insight into cell variation within the tissue, cell identification, cell atlas creation, cellular morphology, identification of foreign entities, like pathogens and viruses, and disease progression mapping.

FISSEQ can detect 3D arranged targets in situ within a matrix, where the detection signal can be a fluorescent signal. Sequencing methods that can be employed by FISSEQ can be sequencing-by-synthesis, sequencing by ligation, or sequencing by hybridization. The targets detected in FISSEQ can be a biomolecule of interest or derivative thereof (e.g., a probe bound to the biomolecule of interest). An example procedure of FISSEQ can comprise contacting a plurality of nucleic acids having a relative 3D spatial relationship within a biological sample with a matrix-forming material to substantially retain the relative 3D spatial relationship; using the matrix-forming material to form a 3D polymerized matrix including the nucleic acids of the plurality of nucleic acids covalently or non-covalently attached to the 3D polymerized matrix; and detecting signals from the nucleic acids or derivatives thereof to identify the nucleic acids. The nucleic acids described herein can be endogenous nucleic acids within the biological sample or synthetic nucleic acids. For example, the endogenous nucleic acids can be attached to the 3D matrix. For another example, the endogenous nucleic acids of may be amplified, for example, by rolling circle amplification (RCA) in situ, and the amplification products can be attached to the 3D matrix before detection. Methods of making a three-dimensional matrix of nucleic acids and amplifying and detecting such nucleic acids within the matrix are disclosed in the U.S. Patent No. U.S. Pat. No. 10,138,509B2, which is entirely incorporated herein by reference.

Targeted FISSEQ generally refers to the detection of a set of targets of interest by FISSEQ using probes targeting the set of targets of interest. For example, probes functioning as reverse transcription primers can be designed to specifically target a set of transcripts of interest instead of targeting all transcripts. Targeted FISSEQ may have the potential for greater per-molecule sensitivity, as cellular volume that would otherwise be occupied by RCA amplicons containing cDNA or DNA sequence irrelevant to a biological phenomenon can be reallocated to the subset of RNA or DNA species of interest. Moreover, for RNA capture, random hexamer priming of reverse transcription may not particularly efficient. In some instances, targeted FISSEQ may have a sensitivity equal to or greater than about 5 times, 10 times, 20 times, 40 times, 80 times, 120 times, 160 times, 200 times, 400 times, 1000 times, 5000 times, or more of that of that of FISSEQ using random hexamer. Moreover, for RNA capture, random hexamer priming of reverse transcription may not be efficient. Other sequence capture methodologies and probe designs may have better capture efficiencies. For DNA capture, targeted capture may also benefit from enhanced per-molecule sensitivity.

Targeted FISSEQ can also be a substantially faster assay than whole transcriptome RNA FISSEQ or whole genome DNA FISSEQ. In some instances, targeted FISSEQ may be about 2 times, 3 times, 4 times, 6 times, 8 times, 10 times, 12 times, 14 times, 16 times, 18 times, or 20 times faster than whole transcriptome RNA FISSEQ or whole genome DNA FISSEQ. As one example, whole-transcriptome FISSEQ may require a sequencing read long enough for high-precision short read alignment. In other words, the sequencing read may need to be long enough to computationally determine the originating molecular species, such as by alignment of the sequencing read to a genomic or transcriptomic reference sequence database. In such whole-omic applications, RNA-seq reads may need to be approximately 20-30 bases long, while genomic reads may need to be longer, such as 50-100 bases long, in order to recover substantially accurate alignments. For targeted FISSEQ of barcode molecular labels, where the barcode labels may be nucleic acid sequences with 4\hat{ }N complexity given a sequencing read of N bases, a much shorter sequencing read may be required for molecular identification. For example, 1024 molecular species may be identified using a 5-nucleotide barcode sequence (4\hat{ }5=1024), whereas 8 nucleotide barcodes can be used to identify up to 65,536 molecular species, a number greater than the total number of distinct genes in the human genome. Therefore, a targeted FISSEQ assay designed to detect each gene in the human transcriptome may be nearly 4× faster (8 bases vs 30 bases), and in the human genome up to more than 12× faster (8 bases vs 100 bases). When targeting specific RNA species for reverse transcription, the space of potential cDNA sequences can be a significant subset of the entire transcriptome, and therefore fewer bases of sequencing are required to identify the target molecule. When targeting specific DNA loci or nucleotides for sequencing or re-sequencing, the space of captured sequences can be a significant subset of the entire genomic sequence or cellular DNA sequence. Targeted FISSEQ where molecular “barcode” sequences contained in the probes can be detected rather than endogenous sequences, can be an efficient read-out in terms of information per cycle of sequencing. Because the barcode sequences are pre-determined, they can also be designed to feature error detection and correction mechanisms. Methods of using targeted FISSEQ and probe designs are disclosed in U.S. patent application Ser. No. 16/285,292, which is entirely incorporated by reference herein.

### FISSEQ for Drug Development Applications

FISSEQ can enable panomic, massively multiplex molecular measurements. Therefore, FISSEQ can create new possibilities for enhancing our understanding of the differences between diseased and healthy biological specimens. Paired measurements of healthy and diseased specimens can enable the curation of molecular targets for drug therapy, including molecules which display a difference in abundance or localization between the healthy and diseased specimens, as well as molecules which may be known or thought to modulate the abundance or localization of those molecules, such as by gene regulatory, developmental, or metabolic signal processing pathways which regulate biological systems. In the same way, other changes, such as in the physical properties of cells and tissues, the composition of cells and tissue, and the organization of cells and tissues, may be used to generate targets for drug development, which may have been known or shown to modulate these properties.

By mapping the disease state onto changes in the molecular profiles of cells and the cellular profile of tissues, FISSEQ can enable stratification of disease states, which may aid in construction of clinical trials, such as by determining cohort composition (as well as providing a basis for subsequent guidance in clinical use).

Finally, FISSEQ can be used for the development of model systems used for drug development, including for development of targeting modalities. For example, FISSEQ can be used to select a target gene, such as one which may be broadly expressed in all cells or in another desired pattern, for RNAi-based therapy development, wherein a library of targeting modalities can be screened for localization of drug-delivery site.

### FISSEQ for Pharmacokinetics Measurements

In situ sequencing, when applied to the detection of therapeutic molecules in situ, can enable significant advances in the study of pharmacokinetics, which is generally understood to be the study of the process of the uptake of drugs by the organism, the biotransformations they undergo, the distribution of the drugs and their metabolites in tissues, and their metabolism and the elimination of the drugs and their metabolites over a period of time.

The panomic nature of FISSEQ can enable direct detection of drugs and their metabolites inside cells and tissues. Nucleic acid drugs may be detected directly using FISSEQ, such as by the fluorescent sequencing of the nucleic acid drug or derivative nucleic acids, such as a biochemically transformed template (e.g., using steps for converting an endogenous nucleic acid into a sequencing template, including but not limited to fragmentation, adaptor ligation, reverse transcription, second-stranding, circularization) and/or as an amplified template (e.g., by generation of an amplicon). Examples of nucleic-acid drugs include, but are not limited to, those used for short interfering RNA mechanisms (siRNA), RNA interference (RNAi), CRISPR/Cas9 and other nucleic-acid-directed nucleic-acid-binding drugs, the nucleic acid aspects of gene therapies, aptamers, ribozymes, anti-sense molecules, decoy molecules, and immunopotentiation drugs. Other types of non-nucleic acid drugs include, but are not limited to, small molecules and biologics (including protein and hormone drugs), and other drugs comprising biomolecular aspects, may be associated with a nucleic acid sequencing template, such as by use of DNA-barcoded affinity binders. Affinity binders used for detection in FISSEQ can include, but are not limited to, antibodies and other classes of natural or engineered immunological proteins, and aptamers.

In certain embodiments, one or more aspects of the drug may be detected using FISSEQ by virtue of the modification or functionalization, such as a sequence or structural modification, PEG modifications, fusion proteins and other protein derivatives, chemical modifications of biomolecules, etc., which may serve to modulate the metabolism or localization of the drugs.

Using FISSEQ, multiplex detection of drugs can be enabled by virtue of the inherent multiplexity of the sequencing assay, where nucleic acids can comprise a vast information encoding space of molecular identity, such as when detected via sequencing by hybridization reactions, including those amplified by HCR, such as the cyclic HCR reaction (CHCR), sequencing by ligation, sequencing by synthesis, and inclusive of all other methods of sequence discrimination using fluorescence signals originating in situ and organized over more than one timepoint of detection.

FISSEQ detection of the spatiotemporal organization of drugs and their metabolites can enable the screening, development, and engineering of the compound or composition related to the localization of the drugs and their metabolites, and the metabolism of the drugs and their metabolites. For example, a FISSEQ pharmacokinetic study may utilize one or more drug compounds featuring a physical-chemical or receptor-mediated delivery mechanism, wherein an aspect of the spatiotemoral localization can be related to the delivery or localization-determining mechanisms, such as for the purpose of measuring and/or improving the specificity of drug localization. Drugs and their metabolites may be directly detected using methods other than FISSEQ, for the purpose of data integration with FISSEQ data collected for the purpose of pharmacodynamic study.

The spatial organization of drugs and their metabolites may be detected with arbitrary spatial resolution, ranging from organ-level localization to sub-cellular localization with nanometer-scale precision, for the purpose of determining the spatiotemporal distribution of therapeutic molecules at tissue, cellular, & sub-cellular spatial scales. Spatiotemporal localization of drugs and their metabolites may be used broadly for the purpose of pharmacokinetic study, including, but not limited to, for detection of localization of the drug to a site of disease, including at the organ or tissue level, at the cellular level, such as to a certain cell type or cell featuring a certain biomolecular profile, and at the sub-cellular level, such as to a site of therapeutic modality, e.g., for inferring the activity by measurement of binding fraction.

### FISSEQ for Pharmacodynamics Measurements

In situ sequencing can also advance the study of pharmacodynamics, which generally refers to the study of the biological response to drugs, including those that are intended and untended, therapeutic, detrimental, or neutral, over a period of time, such as during the course of treatment and after treatment is suspended. The effects of drug action may typically fall into one of a number of categories, including, but not limited to: stimulating action, such as through direct receptor agonism and downstream effects; depressing action, such as through direct receptor agonism and downstream effects (e.g., an inverse agonist); blocking or antagonizing action, such as when the drug binds the receptor but does not activate it; stabilizing action, such as when the drug seems to act neither as a stimulant or as a depressant. The effects of drug action can be achieved by exchanging or replacing substances or accumulating them to form a reserve, by direct beneficial chemical reaction, such as in free radical scavenging, or by direct harmful chemical reaction, which might result in damage or destruction of the cells, such as through induced toxic or lethal damage. Moreover, these effects can be mediated by one of a number of mechanisms at the molecular level, including, but not limited to, interaction with proteins, such as enzymes, structural proteins, carriers, transport or ion channels, signaling proteins and receptors; ligand binding; and other mechanisms such as by disruption of lipid or membrane structures, by interactions with nucleic acids, or by other chemical reactions.

Pharmacodynamics can also be concerned with the quantitative response to drug therapy, including the therapeutic window, the aspect of dose-response concerned with the difference between the minimum effective dose and the dose at which adverse effects occur, as well as the temporal aspect of drug therapy, such as the duration of activity.

FISSEQ can enable direct measurement of drugs' molecular mechanisms and effects. Given the panomic, massively multiplex nature of the FISSEQ technology, it may be possible to simultaneously measure the effect of drug treatment on the abundance and localization of a variety of molecular targets. Molecular targets may include those involved in the disease mechanism or other markers of desired response, mechanisms of toxicity or undesired responses, as well as control genes, which are thought to be independent of drug treatment in their abundance and localization, which may be used for the purpose of data normalization or other statistical interpretation of the FISSEQ data. RNA, protein, DNA, and small molecule targets may be assayed. According to one aspect, the assay may be wholly-omic, or un-targeted, such as the randomly-primed RNA-FISSEQ assay.

FISSEQ can also enable direct measurement of drugs' cellular, tissue, organ, and organism-level phenotypic responses, such as changes in the composition or spatial organization of these features. For example, using FISSEQ it may be possible to detect changes in tissue architecture, such as changes in the composition or organization of cell types and cellular molecular expression profiles and activity, within the tissue or organ. FISSEQ can be used to detect changes in cell-cell signaling, such as by detecting metabolites or signaling molecules, as well as by detecting receptors and their states, and other intracellular and extracellular components of receptor-mediated signaling. FISSEQ can be used to detect changes in the composition and organization of the extracellular matrix, the structural and transport environment surrounding cells within an organism.

FISSEQ can also enable inference drugs' molecular mechanisms and effects. By co-detection of drugs and their metabolites with detection of endogenous molecules, such as RNA, DNA, and protein, it may be possible to infer binding and other proximity-mediated reactions. For example, FISSEQ libraries may be constructed for co-detection and/or proximity detection of two or more analytes for the purpose of measuring the binding properties of a drug.

By using FISSEQ assays with sufficient sequence resolution, it may be possible to measure changes in the sequence of nucleic acid molecules (e.g., genetic aberrations) resulting from drug treatment, such as allele-specific expression, alternative splicing and exon-skipping, and mutations or engineered changes in sequence.

Drug effects may be desirable or undesirable. Undesirable effects can include, but are not limited to, increased probability of mutation, interactions, such as with other drugs or genetic or environmental background, and other deleterious or damaging effects. FISSEQ can be used to detect accumulation of therapeutic compounds in compartments relevant to toxicity and other undesired effects, immune and cellular immune responses, sensitization, and other undesired effects.

Furthermore, by inclusively measuring the molecular changes occurring in response to drug treatment, it may be possible to guide therapeutic development, such as by using an adjuvant or secondary drug to enhance a desired effect or remediate an undesired effect.

### Data Integration

FISSEQ data for the purposes described herein may be combined with other types of information, such as bulk and/or single-cell analyte detection. FISSEQ assay of one or more drug(s) or drug composition(s) may be combined with assay of endogenous biomolecules, enabling powerful characterization of drugs encompassing both their pharmacokinetic and pharmacodynamic properties.

### Therapeutic Agent and Molecular Target

Provided herein are methods and systems for detection of therapeutic agents and molecular targets thereof. The molecular targets may be any molecule of interest in a biological sample which may or may not be bound to a therapeutic agent directly. The molecular target can have a function in a disease. In some cases, the molecular target may be bound to a therapeutic agent directly, and the molecular target and its therapeutic agent can co-localize within the biological sample. In some cases, the molecular target may be downstream of a signaling pathway affected by an upstream molecule bound directly by a therapeutic agent, and therefore the molecular target may not be bound to a therapeutic agent directly.

The therapeutic agent can be a nucleic acid therapeutic agent, a protein therapeutic agent, a peptide therapeutic agent, or a small molecule therapeutic agent. In various cases, the nucleic acid therapeutic agent or derivative thereof (e.g., an amplification product of the nucleic acid sequence of the nuclei acid therapeutic agent or a probe specifically targeting the nucleic acid therapeutic agent) can be detected by FISSEQ as described herein. In the cases where the therapeutic agent does not comprise a nucleic acid sequence, such as the protein therapeutic agent, the peptide therapeutic agent or the small molecule therapeutic agent, the therapeutic agent may be linked to a nucleic acid sequence for detection via FISSEQ as described herein.

In some cases, the molecular target may be a nucleic acid target. The nucleic acid target can be a ribonucleic acid (RNA) or a deoxyribonucleic acid (DNA). The nucleic acid target may be single-stranded or double-stranded. The nucleic acid target may be naturally occurring nucleic acids or non-naturally occurring nucleic acids, such as nucleic acids that have been made using synthetic methods. The nucleic acid target can be an endogenous nucleic acid in a biological sample, for example, genomic DNA, messenger RNA (mRNA), ribosomal RNA (rRNA), transfer RNA (tRNA), microRNA (miRNA), small cytoplasmic RNA (scRNA), and small nuclear RNA (snRNA). The therapeutic agent can bind to the nucleic acid target directly. The therapeutic agent can affect the functions or expression levels of the nucleic acid target. In some cases, the therapeutic agent may not bind to the nucleic acid target directly but affect the functions or expression level of the nucleic acid target. For example, the therapeutic agent which binds to a nucleic acid target can be an anti-sense oligonucleotide. As used herein, the “anti-sense oligonucleotide” refers to a nucleic acid sequence that comprises a reverse complement of a sequence of the nucleic acid target of interest. The anti-sense oligonucleotide can bind to the nucleic acid target and affect its structure or function. The binding of the anti-sense oligonucleotide to the active target gene or its transcripts can cause decreased expression through a variety of processes. Binding can occur either through the blocking of transcription (in the case of gene-binding), the degradation of the mRNA transcript (e.g., by small interfering RNA (siRNA)) or RNase-H dependent anti-sense), or through the blocking of either mRNA translation, pre-mRNA splicing sites, or nuclease cleavage sites used for maturation of other functional RNAs, including miRNA (e.g., by morpholino oligos or other RNase-H independent anti-sense). The anti-sense oligonucleotide can be single-stranded or double-stranded. Examples of anti-sense oligonucleotide include RNase-H dependent anti-sense oligos, small interfering RNA (siRNA), miRNA, and short hairpin RNA (shRNA). The anti-sense oligonucleotide may be DNA. The nucleic acid target may be bound by a protein therapeutic agent (e.g., a nucleic acid-binding protein), and in such cases, the protein therapeutic agent can be linked with a nucleic acid sequence directly or indirectly. The nucleic acid sequence directly or indirectly linked to the protein therapeutic agent can be detected by FISSEQ.

In some cases, the molecular target may be a protein. In the cases where the molecular target is a protein, a therapeutic agent (e.g., antibody or a fragment thereof and small molecule) which binds to the protein can be linked to a nucleic acid sequence which can then be detected by the methods and systems provided herein. In some cases, a therapeutic agent for a protein target can be a protein therapeutic agent. Examples of protein therapeutic agent include, but are not limited to, monoclonal antibodies, fusion proteins (e.g., Fc-fusion proteins), cytokines and hormones. In some cases, a therapeutic agent for a protein target can be an aptamer. In some other cases, a therapeutic agent for a protein target can be a small molecule.

For small molecule detection, a DNA-barcoded affinity binding reagent specific to the small molecule can be generated. Another method may be to develop dynamic small-molecule biosensors, which can undergo conformational changes in the presence of the target ligand. Biosensors as used herein may refer to genetically encoded biosensors that modulate gene expression in response to the presence of a small molecule inducer. Biosensors may be a part of small molecule inducible systems comprising genetically encoded biosensors. Such biosensor system can transfer the activity or abundance of small molecules into the transcription level of certain RNA species through transcriptional repression or activation. For example, the biosensors can be proteins, where the proteins function as transcriptional repressors or activators. In some cases, the transcription repressors or activators can be regulated by small molecules, which in turn regulate RNA transcription. In such cases, the abundance and/or presence of certain RNA transcripts/species can be used to determine the level and/or presence of regulatory small molecules.

In order to retain the small molecules (e.g., therapeutic agents or metabolites) in situ for detection, chemistries to cross link small molecules to an expanding hydrogel matrix need to be developed, which enables permeabilization of the sample by dilution of the biomolecules during expansion. Moreover, as with calcium imaging, FISSEQ experiments that blur the line between in situ and in vivo can be designed. For example, fluorescent biosensors can be used to measure the dynamics of metabolite abundance and localization in vivo, which can be combined with a single time point measurement of gene expression or genotype in situ. Biosensors can also record the abundance and localization of small molecules into RNA, as by activating transcription upon binding, or by directly encoding this information into the genome, such as by using CRISPR/Cas9 genome editing technology. In the former case, the RNA molecules containing the information about the metabolite concentration in vivo may be detected in situ using FISSEQ. In the latter case, the modified genome sequence may be detected in situ using FISSEQ.

A nucleic acid sequence, from either nucleic acid target or a therapeutic agent, can be detected or analyzed using FISSEQ as described herein. The nucleic acid molecule having the nucleic acid sequence can be present within a three-dimensional (3D) matrix and covalently attached to the 3D matrix such that the relative position of each nucleic acid is fixed (e.g., immobilized) within the 3D matrix. In this manner, a 3D matrix of covalently bound nucleic acids of any sequence can be provided. Each nucleic acid may have its own three-dimensional coordinates within the matrix material and each nucleic acid may represent information. In this manner, a large amount of information can be stored in a 3D matrix. Individual information-encoding nucleic acid target, such as DNA or RNA can be amplified and sequenced in situ (i.e., within the matrix), thereby enabling a large amount of information to be stored and read in a suitable 3D matrix.

The nucleic acid molecule (either a molecular target or a therapeutic agent) may be amplified to produce amplification products or amplicons within the 3D matrix. The nucleic acid target may be amplified using nucleic acid amplification, such as, for example, polymerase chain reaction (PCR). The nucleic acid molecule may be bound to a probe (e.g., a detection probe) and the probe may be subsequently amplified to produce amplification products or amplicons. In some cases, the nucleic acid molecule is an RNA molecule, and the RNA molecule may be reverse transcribed to generate a cDNA. The probe (e.g., a detection probe) used to bind to the target RNA molecule can function as a reverse transcription primer. The cDNA may then be subjected to amplification or may be contacted with a probe. The amplification products or amplicons can be attached to the matrix, for example, by copolymerization or cross-linking. This can result in a structurally stable and chemically stable 3D matrix of nucleic acids. The 3D matrix of nucleic acids may allow for prolonged information storage and read-out cycles. The nucleic acid/amplicon matrix may allow for high throughput sequencing of a wide ranging array of samples in three dimensions. In some cases, the RNA molecule may be directly targeted by a probe without reverse transcription. The probe may be directly or indirectly labeled with a reporter agent for detection as described herein.

### Methods for Analysis

Provided herein are methods and systems for analyzing or identifying an agent in a biological sample. The agent can be a therapeutic agent, for example, an anti-sense ribonucleic acid (RNA) and a small molecule inhibitor.

In some embodiments, the present disclosure provides a method for identifying an anti-sense nucleic acid molecule and a target molecule. The method can comprise providing a biological sample having or suspected of having the target molecule and the anti-sense nucleic acid molecule. The biological sample can comprise a three-dimensional (3D) matrix. Next, detection probes separate from the anti-sense nucleic acid molecule and the target molecule can be used to detect a first set of signals and a second set of signals from the biological sample. The first set of signals can identify a relative position of the anti-sense nucleic acid molecule in the biological sample. The second set of signals can identify an increase or a decrease in a level of the target molecule relative to a reference. Next, the first set of signals and the second set of signals can be used to provide an output indicative of a position of each of the anti-sense nucleic acid molecule and the target molecule in the biological sample.

In some cases, the method may further comprise, prior to providing the biological sample, contacting the biological sample with a solution having the anti-sense nucleic acid molecule. Next, the 3D matrix can be generated subsequent to contacting the biological sample with the solution.

The target molecule can be a target nucleic acid molecule. The target nucleic acid molecule can be a deoxyribonucleic acid (DNA) molecule or a ribonucleic acid (RNA) molecule. The detection probes can be used to identify a target sequence of the target nucleic acid molecule, thereby providing the second set of signals. The detection probes can be used to sequence the target nucleic acid molecule, thereby identifying the target sequence. The detection probes can be used to identify an anti-sense sequence of the anti-sense nucleic acid molecule, thereby providing the first set of signals. The detection probes can be used to sequence the anti-sense nucleic acid molecule, thereby identifying the anti-sense sequence.

The target molecule can be a target polypeptide or protein. The biological sample can be a cell, a cell derivative, or a tissue. The anti-sense nucleic acid molecule can be a therapeutic agent. The output can be an image or video. The anti-sense nucleic acid molecule can be a ribonucleic acid, a phosphorothioate deoxyribonucleic acid, a locked nucleic acid, 2′-O-methocy-ethyl ribonucleic acid, 2′-O-methyl ribonucleic acid, or a 2′-fluoro deoxyribonucleic acid.

The anti-sense nucleic acid molecule can be from 15 to 20, from 20 to 30, from 30 to 40, from 40 to 50, or from 50 to 60 nucleotides in length. In some cases, the anti-sense nucleic acid molecule can be at least about 5, 10, 15, 20, 25, 30, 35, 40, 45, 50, or more nucleotides in length.

In some embodiments, the present disclosure provides a method for identifying an anti-sense ribonucleic acid (RNA) molecule co-localized with a target RNA molecule. The method can comprise providing a biological sample having or suspected of having the target RNA molecule and the anti-sense RNA molecule of the target RNA molecule. The biological sample can comprise a three-dimensional (3D) matrix. Next, a first set of signals and a second set of signals from the biological sample can be detected. The first set of signals can identify a relative position of the anti-sense RNA molecule in the biological sample. The second set of signals can identify an increase or a decrease in a level of the target RNA molecule relative to a reference. The first set of signals and the second set of signals can be used to provide an output indicative of the anti-sense RNA molecule co-localized with the target RNA molecule in the biological sample.

In some cases, the method may further comprise, prior to providing the biological sample, contacting the biological sample with a solution having the anti-sense RNA molecule. Next, the 3D matrix can be generated subsequent to contacting the biological sample with the solution.

The biological sample can be a cell, a cell derivative, or a tissue. The detection probes can be used to identify an anti-sense sequence of the anti-sense RNA molecule, thereby providing the first set of signals. The detection probes can be used to sequence the anti-sense RNA molecule, thereby identifying the anti-sense sequence. The detection probes can be used to identify a target sequence of the target RNA molecule, thereby providing the second set of signals. The detection probes can be used to sequence the target RNA molecule, thereby identifying the target sequence.

The anti-sense RNA molecule can be a therapeutic agent. The output can be an image or video. The anti-sense RNA molecule can be a modified anti-sense nucleic acid molecule. The modified anti-sense nucleic acid molecule can be a phosphorothioate deoxyribonucleic acid, a locked nucleic acid, 2′-O-methocy-ethyl ribonucleic acid, 2′-O-methyl ribonucleic acid, or a 2′-fluoro deoxyribonucleic acid. The anti-sense RNA molecule can be from 15 to 20, from 20 to 30, from 30 to 40, from 40 to 50, or from 50 to 60 nucleotides in length. In some cases, the anti-sense RNA molecule can be at least about 5, 10, 15, 20, 25, 30, 35, 40, 45, 50, or more nucleotides in length.

In some embodiments, the present disclosure provides a method for identifying two or more genetic aberrations of a target nucleic acid. The method can comprise providing a biological sample having or suspected of having at least a first genetic aberration and a second genetic aberration of the target nucleic acid. The biological sample can comprise a three-dimensional (3D) matrix. Next, detection probes can be used to detect a first set of signals and a second set of signals from the biological sample. The first set of signals can identify a first relative position of the first genetic aberration in the biological sample. The second set of signals can identify a second relative position of the second genetic aberration in the biological sample. Next, the first set of signals and the second set of signals can be used to provide an output indicative of a position of each of the first genetic aberration and the second genetic aberration in the biological sample.

The detection probes may be targeted to a same sequence of the first genetic aberration and the second genetic aberration. The first genetic aberration and the second genetic aberration may have at least a single nucleotide difference. The first genetic aberration can comprise an additional sequence than the second genetic aberration. The biological sample can be a cell, a cell derivative, or a tissue.

In some embodiments, the present disclosure provides a method for identifying two or more locations of an anti-sense nucleic acid molecule. The method can comprise providing a biological sample having the anti-sense nucleic acid molecule. The biological sample can comprise a three-dimensional (3D) matrix. Next, a detection probe can be used to detect a set of signals of the anti-sense nucleic acid molecule within the biological sample. A first set of signals and a second set of signals can be detected from at least a first location and a second location within the biological sample. The first location and the second location can be within different sub-cellular compartments. The set of signals of the anti-sense nucleic acid molecule and the first and the second set of signals of the first location and the second location can be used to provide an output indicative of the anti-sense nucleic acid molecule co-localized with the first location and the second location.

The anti-sense nucleic acid molecule can be from 15 to 20, from 20 to 30, from 30 to 40, from 40 to 50, or from 50 to 60 nucleotides in length. In some cases, the anti-sense nucleic acid molecule can be at least about 5, 10, 15, 20, 25, 30, 35, 40, 45, 50, or more nucleotides in length. The sub-cellular compartments can comprise mitochondria, endoplasmic reticulum, P-bodies, Golgi apparatus, vesicle, endosome, lysosome, nucleus, microtubule, or a combination thereof. The anti-sense nucleic acid molecule can be a therapeutic agent. The biological sample can be a cell, a cell derivative, or a tissue.

In some embodiments, the present disclosure provides a method for identifying co-localized molecules in a biological sample. The method can comprise providing the biological sample having or suspected of having a target molecule and an agent capable of binding or interacting with the target molecule. The biological sample can comprise a three-dimensional (3D) matrix. Next, detection probes separate from the target molecule and the agent can be used to detect a first set of signals and a second set of signals from the biological sample. The first set of signals can identify a relative position of the target molecule in the biological sample. The second set of signals can identify a relative position of the agent in the biological sample. The first set of signals and the second set of signals can be used to provide an output indicative of the target molecule co-localized with the agent in the biological sample.

The agent can be a therapeutic agent. The therapeutic agent can be a polynucleotide, a polypeptide, or a small molecule.

In some embodiments, the present disclosure provides a method for identifying an agent and a metabolite of the agent in a biological sample. The method can comprise providing the biological sample having or suspected of having the agent and the metabolite of the agent. The biological sample can comprise a three-dimensional (3D) matrix. Next, detection probes separate from the target molecule and the agent can be used to detect a first set of signals and a second set of signals from the biological sample. The first set of signals can identify a relative position of the agent in the biological sample. The second set of signals can identify a relative position of the metabolite in the biological sample. Next, the first set of signals and the second set of signals can be used to provide an output indicative of a position of each of the agent and the metabolite. The biological sample can be a cell, a cell derivative, or a tissue. The agent can be a therapeutic agent. The therapeutic agent can be a polynucleotide, a polypeptide, or a small molecule.

### Three-Dimensional Matrix

The present disclosure provides a three-dimensional (3D) matrix. The 3D matrix may comprise a plurality of nucleic acids. The 3D matrix may comprise a plurality of nucleic acids covalently or non-covalently attached thereto.

In some cases, a matrix-forming material may be used to form the 3D matrix. The matrix forming material may be polymerizable monomers or polymers, or cross-linkable polymers. The matrix forming material may be polyacrylamide, acrylamide monomers, cellulose, alginate, polyamide, agarose, dextran, or polyethylene glycol. The matrix forming materials can form a matrix by polymerization and/or crosslinking of the matrix forming materials using methods specific for the matrix forming materials and methods, reagents and conditions. The matrix forming material may form a polymeric matrix. The matrix forming material may form a polyelectrolyte gel. The matrix forming material may form a hydrogel gel matrix.

The matrix-forming material may form a 3D matrix including the plurality of nucleic acids while maintaining the spatial relationship of the nucleic acids. In this aspect, the plurality of nucleic acids can be immobilized within the matrix material. The plurality of nucleic acids may be immobilized within the matrix material by co-polymerization of the nucleic acids with the matrix-forming material. The plurality of nucleic acids may also be immobilized within the matrix material by crosslinking of the nucleic acids to the matrix material or otherwise cross-linking with the matrix-forming material. The plurality of nucleic acids may also be immobilized within the matrix by covalent attachment or through ligand-protein interaction to the matrix.

According to one aspect, the matrix can be porous thereby allowing the introduction of reagents into the matrix at the site of a nucleic acid for amplification of the nucleic acid. A porous matrix may be made according to various methods. For example, a polyacrylamide gel matrix can be co-polymerized with acrydite-modified streptavidin monomers and biotinylated DNA molecules, using a suitable acrylamidebis-acrylamide ratio to control the cross-linking density. Additional control over the molecular sieve size and density can be achieved by adding additional cross-linkers such as functionalized polyethylene glycols.

According to one aspect, the 3D matrix may be sufficiently optically transparent or may have optical properties suitable for standard sequencing chemistries and deep three-dimensional imaging for high throughput information readout. Examples of the sequencing chemistries that utilize fluorescence imaging include ABI SoLiD (Life Technologies), in which a sequencing primer on a template is ligated to a library of fluorescently labeled octamers with a cleavable terminator. After ligation, the template can then be imaged using four color channels (FITC, Cy3, Texas Red and Cy5). The terminator can then be cleaved off leaving a free-end to engage in the next ligation-extension cycle. After all dinucleotide combinations have been determined, the images can be mapped to the color code space to determine the specific base calls per template. The workflow can be achieved using an automated fluidics and imaging device (i.e., SoLiD 5500 W Genome Analyzer, ABI Life Technologies). Another example of sequencing platform uses sequencing by synthesis, in which a pool of single nucleotide with a cleavable terminator can be incorporated using DNA polymerase. After imaging, the terminator can be cleaved, and the cycle can be repeated. The fluorescence images can then be analyzed to call bases for each DNA amplicons within the flow cell (HiSeq, Illumia).

In some aspects, a biological sample may be fixed in the presence of the matrix-forming materials, for example, hydrogel subunits. By “fixing” the biological sample, it is meant exposing the biological sample, e.g., cells or tissues, to a fixation agent such that the cellular components become crosslinked to one another. By “hydrogel” or “hydrogel network” is meant a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may comprise natural or synthetic polymers, or a combination thereof. Hydrogels may also possess a degree of flexibility very similar to natural tissue, due to their significant water content. By “hydrogel subunits” or “hydrogel precursors” refers to hydrophilic monomers, prepolymers, or polymers that can be crosslinked, or “polymerized”, to form a 3D hydrogel network. Without being bound by any scientific theory, fixation of the biological sample in the presence of hydrogel subunits may crosslink the components of the biological sample to the hydrogel subunits, thereby securing molecular components in place, preserving the tissue architecture and cell morphology.

In some cases, the biological sample may be fixed and/or permeabilized first, and then a matrix-forming material can then be added into the biological sample.

Any convenient fixation agent, or “fixative,” may be used to fix the biological sample in the absence or in the presence of hydrogel subunits, for example, formaldehyde, paraformaldehyde, glutaraldehyde, acetone, ethanol, methanol, etc. Typically, the fixative may be diluted in a buffer, e.g., saline, phosphate buffer (PB), phosphate buffered saline (PBS), citric acid buffer, potassium phosphate buffer, etc., usually at a concentration of about 1-10%, e.g. 1%, 2%, 3%, 4%, 5%, 6%, 7%, 8%, or 10%, for example, 4% paraformaldehyde/0.1M phosphate buffer; 2% paraformaldehyde/0.2% picric acid/0.1M phosphate buffer; 4% paraformaldehyde/0.2% periodate/1.2% lysine in 0.1 M phosphate buffer; 4% paraformaldehyde/0.05% glutaraldehyde in phosphate buffer; etc. The type of fixative used and the duration of exposure to the fixative will depend on the sensitivity of the molecules of interest in the specimen to denaturation by the fixative, and may be readily determined using conventional histochemical or immunohistochemical techniques.

The fixative/hydrogel composition may comprise any hydrogel subunits, such as, but not limited to, poly(ethylene glycol) and derivatives thereof (e.g. PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. Agents such as hydrophilic nanoparticles, e.g., poly-lactic acid (PLA), poly-glycolic acid (PLG), poly(lactic-co-glycolic acid) (PLGA), polystyrene, poly(dimethylsiloxane) (PDMS), etc. may be used to improve the permeability of the hydrogel while maintaining patternability. Materials such as block copolymers of PEG, degradable PEO, poly(lactic acid) (PLA), and other similar materials can be used to add specific properties to the hydrogel. Crosslinkers (e.g. bis-acrylamide, diazirine, etc.) and initiators (e.g. azobisisobutyronitrile (AIBN), riboflavin, L-arginine, etc.) may be included to promote covalent bonding between interacting macromolecules in later polymerization steps.

The biological sample (e.g., a cell or tissue) may be permeabilized after being fixed. Permeabilization may be performed to facilitate access to cellular cytoplasm or intracellular molecules, components or structures of a cell. Permeabilization may allow an agent (such as a phospho-selective antibody, a nucleic acid conjugated antibody, a nucleic acid probe, a primer, etc.) to enter into a cell and reach a concentration within the cell that is greater than that which may normally penetrate into the cell in the absence of such permeabilizing treatment. In some embodiments, cells may be stored following permeabilization. In some cases, the cells may be contacted with one or more agents to allow penetration of the one or more agent after permeabilization without any storage step and then analyzed. In some embodiments, cells may be permeabilized in the presence of at least about 60%, 70%, 80%, 90% or more methanol (or ethanol) and incubated on ice for a period of time. The period of time for incubation can be at least about 10, 15, 20, 25, 30, 35, 40, 50, 60 or more minutes.

In some embodiments, permeabilization of the cells may be performed by any suitable method. Selection of an appropriate permeabilizing agent and optimization of the incubation conditions and time may be performed. Suitable methods include, but are not limited to, exposure to a detergent (such as CHAPS, cholic acid, deoxycholic acid, digitonin, n-dodecyl-beta-D-maltoside, lauryl sulfate, glycodeoxycholic acid, n-lauroylsarcosine, saponin, and triton X-100) or to an organic alcohol (such as methanol and ethanol). Other permeabilizing methods can comprise the use of certain peptides or toxins that render membranes permeable. Permeabilization may also be performed by addition of an organic alcohol to the cells.

Permeabilization can also be achieved, for example, by way of illustration and not limitation, through the use of surfactants, detergents, phospholipids, phospholipid binding proteins, enzymes, viral membrane fusion proteins and the like; through the use of osmotically active agents; by using chemical crosslinking agents; by physicochemical methods including electroporation and the like, or by other permeabilizing methodologies.

Thus, for instance, cells may be permeabilized using any of a variety of techniques, such as exposure to one or more detergents (e.g., digitonin, Triton X-100™, NP-40™, octyl glucoside and the like) at concentrations below those used to lyse cells and solubilize membranes (i.e., below the critical micelle concentration). Certain transfection reagents, such as dioleoyl-3-trimethylammonium propane (DOTAP), may also be used. ATP can also be used to permeabilize intact cells. Low concentrations of chemicals used as fixatives (e.g., formaldehyde) may also be used to permeabilize intact cells.

The nucleic acids (e.g., RNA molecule, cDNA molecule, primer, or probe) described herein may comprise a functional moiety. The nucleic acids can be linked to the 3D matrix by the functional moiety. The functional moiety can be reacted with a reactive group on the 3D matrix through conjugation chemistry. In some cases, the functional moiety can be attached to target of interest through conjugation chemistry. In some cases, the functional moiety can be directly attached to a reactive group on the native nucleic acid molecule. In some cases, the functional moiety can be indirectly linked to a target through an intermediate chemical or group. The conjugation strategies described herein are not limited to nucleic acid targets and can be used for protein or small molecule targets as well. A nucleotide analog comprising a functional moiety may be incorporated into a growing chain of the nucleic acid (e.g., cDNA molecule, probe, or primer) during nucleic acid synthesis or an extension reaction.

The present disclosure provides methods for modifying a nucleic acid in situ to comprise a functional moiety. In some cases, the functional moiety may comprise a polymerizeable group. In some cases, the functional moiety may comprise a free radical polymerizeable group. In some cases, the functional moiety may comprise an amine, a thiol, an azide, an alkyne, a nitrone, an alkene, a tetrazine, tetrazole, or other click reactive group. In some cases, the functional moiety can be subsequently linked to a 3D matrix in situ. The functional moiety may further be used to preserve the absolute or relative spatial relationships among two or more molecules within a sample.

### Support

A matrix may be used in conjunction with a support. The support may be a solid or semi-solid support. For example, the matrix can be polymerized in such a way that one surface of the matrix is attached to a support (e.g., a glass surface, a flow cell, a glass slide, a well), while the other surface of the matrix is exposed. Alternatively, the matrix can be sandwiched between two supports. According to some aspects, the matrix can be contained within a container. In some cases, the biological sample may be fixed or immobilized on a support.

Supports of the present disclosure may be fashioned into a variety of shapes. In certain embodiments, the support is substantially planar. Examples of supports include plates such as slides, microtiter plates, flow cells, coverslips, microchips, and the like, containers such as microfuge tubes, test tubes and the like, tubing, sheets, pads, films and the like. Additionally, the supports may be, for example, biological, nonbiological, organic, inorganic, or a combination thereof.

A support can be made of any material that can serve as a solid or semi-solid foundation for attachment of a biological sample or molecules such as polynucleotides, amplicons, DNA balls, and/or polymers, including biopolymers. Example types of materials include, but are not limited to, glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, and multiwell microtiter plates. Specific types of example plastics include acrylics, polystyrene, copolymers of styrene and other materials, polypropylene, polyethylene, polybutylene, polyurethanes and Teflon™. Specific types of example silica-based materials include silicon and various forms of modified silicon.

The surface of a support can be planar or contain regions which are concave or convex.

### Detection Probes

The nucleic acid sequences described herein (e.g., target nucleic acid molecules, therapeutic agents, or nucleic acid sequences linked to therapeutic agents) can be bound by detection probes. The detection probes can be used for subsequent detection of the nucleic acid sequences of interest such as by imaging or sequencing.

A detection probe can be a nucleic acid probe. A detection probe may be a padlock probe, a molecular inversion probe, a molecular beacon probe, a reverse transpiration primer, a second strand synthesis primer or other primers used for nucleic acid synthesis (e.g., amplification) described herein. A detection probe may preferentially bind a sequence over another sequence. A detection probe may emit a signal when hybridized to a sequence to allow identification of the sequence and/or the identification of a particular location. A detection probe may be directly linked to a reporter agent. A detection probe may not be directly linked to a report agent. A detection probe may be used to synthesize or amplify nucleic acids which may be subjected to sequencing reactions as described herein. A detection probe may be bound by an additional probe for detection.

The detection probe may be ribonucleic acid, deoxyribonucleic acid, or derivatives thereof, or any combinations thereof. The detection probe may be of a particular length. The detection probe may be less than or equal to about 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, or 2 nucleotides long. The detection probe may be greater than or equal to about 2, 3, 4, 5, 6, 7, 8, 9, 10, 11, 12, 13, 14, 15, 16, 17, 18, 19, 20, 25, 30, 35, 40, 50, or more nucleotides long. The detection probe may be of any configurations, including but not limited to linear, circular, and stem-loop.

The detection probe can comprise a barcode. The barcode can be a unique molecule identifier. In some cases, a pool of detection probes are used for FISSEQ detection, each probe of the pool has a unique barcode sequence. The barcode can be at least about 2, 3, 4, 5, 6, 7, 8, 9, 10, 11, 12, 13, 14, 15, 16, 17, 18, 19, 20, 21, 22, 23, 24, 25, 26, 27, 29, 30, or more nucleotides in length.

In some cases, the detection probe may be a protein probe. For example, in some cases, the target to be detected is a protein, and the probe molecules may be antibodies, fragments of antibodies, or derivatives of antibodies, which can bind to the protein target. The detection probe may also be a nucleic acid binding protein. The nucleic acid binding protein may bind preferentially to a specific sequence. The nucleic acid binding protein may bind non-specifically. The nucleic acid proteins may bind a specific nucleotide or nucleotide derivate or may bind a particular structure of nucleic acid.

### Amplification

In some cases, a nucleic acid target or derivative thereof may be amplified. For example, an RNA target molecule may be reverse transcribed to generate a cDNA molecule, and the cDNA molecule may be amplified in situ. Any type of nucleic acid amplification reaction may be used to perform an amplification reaction in the methods or systems described herein and generate an amplification product. Moreover, amplification of a nucleic acid may be linear, exponential, or a combination thereof. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In some cases, the amplified product may be DNA. In cases where a target RNA is amplified, DNA can be obtained by reverse transcription of the RNA and subsequent amplification of the DNA can be used to generate an amplified DNA product. The amplified DNA product may be indicative of the presence of the target RNA in the biological sample. In cases where DNA is amplified, any DNA amplification method may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products.

The amplification of nucleic acid sequences may be performed within the matrix. Methods of amplifying nucleic acids may include rolling circle amplification in situ. In certain aspects, methods of amplifying nucleic acids may include the use of PCR, such as anchor PCR, RACE PCR, or a ligation chain reaction (LCR). Alternative amplification methods include but are not limited to self-sustained sequence replication, transcriptional amplification system, Q-Beta Replicase, recursive PCR or any other nucleic acid amplification method.

The nucleic acids within the 3D matrix may be contacted with reagents under suitable reaction conditions sufficient to amplify the nucleic acids. The matrix may be porous to allow migration of reagents into the matrix to contact the nucleic acids. In certain aspects, nucleic acids may be amplified by selectively hybridizing an amplification primer to an amplification site at the 3′ end of a nucleic acid sequence using conventional methods. Amplification primers can be from 6 to 100, and even up to 1,000, nucleotides in length, but typically from 10 to 40 nucleotides, although oligonucleotides of different length are of use. In some cases, the amplification primer may be at least about 5, 6, 7, 8, 9, 10, 11, 12, 13, 14, 15, 16, 17, 19, 20, 25, 30, 35, 40, 45, 50, or more nucleotides in length. In some cases, the amplification primer may be at least about 50, 55, 60, 65, 70, 75, 80, 85, 90, 95, 100, 150, 200, 250, 300, or more nucleotide in length. Amplification primers may hybridize to a nucleic acid probe that hybridizes to a DNA molecule such that the amplification primers can be used to amplify a sequence of the nucleic acid probe. Amplification primers may be present in solution to be added to the matrix or they may be added during formation of the matrix to be present therein sufficiently adjacent to nucleic acids to allow for hybridization and amplification.

A DNA polymerase can be used in an amplification reaction. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof.

### Detection

The present disclosure provides methods and systems for sample processing for use in nucleic acid detection. A sequence of the nucleic acid target may be identified. Various methods can be used for nucleic acid detection, including hybridization, imaging and sequencing.

Reporter agents may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. Reporter agents may bind to initial reactants and changes in reporter agent levels may be used to detect amplified product. Reporter agents may be detectable (or non-detectable) as nucleic acid amplification progresses. Reporter agents may be optically detectable. An optically-active dye (e.g., a fluorescent dye) may be used as a reporter agent. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a reporter agent may be a sequence-specific oligonucleotide probe that is optically active when hybridized with a nucleic acid target or derivative thereof (e.g., an amplified product). A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

In some aspects, the method for determining the nucleic acid sequence of a target nucleic acid molecule includes sequencing. In some aspects, sequencing by synthesis, sequencing by ligation or sequencing by hybridization is used for deterring the nucleic acid sequence of a target nucleic acid molecule. As disclosed herein, various amplification methods can be employed to generate larger quantities, particularly of limited nucleic acid samples, prior to sequencing. For example, the amplification methods can produce a targeted library of amplicons.

For sequencing by ligation, labeled nucleic acid fragments may be hybridized and identified to determine the sequence of a target nucleic acid molecule. For sequencing by synthesis (SBS), labeled nucleotides can be used to determine the sequence of a target nucleic acid molecule. A target nucleic acid molecule can be hybridized with a primer and incubated in the presence of a polymerase and a labeled nucleotide containing a blocking group. The primer can be extended such that the nucleotide is incorporated. The presence of the blocking group may permit only one round of incorporation, that is, the incorporation of a single nucleotide. The presence of the label can permit identification of the incorporated nucleotide. As used herein, a label can be any optically active dye described herein. Either single bases can be added or, alternatively, all four bases can be added simultaneously, particularly when each base is associated with a distinguishable label. After identifying the incorporated nucleotide by its corresponding label, both the label and the blocking group can be removed, thereby allowing a subsequent round of incorporation and identification. Thus, it is desirable to have conveniently cleavable linkers linking the label to the base, such as those disclosed herein, in particular peptide linkers. Additionally, it is advantageous to use a removable blocking group so that multiple rounds of identification can be performed, thereby permitting identification of at least a portion of the target nucleic acid sequence. The compositions and methods disclosed herein are particularly useful for such an SBS approach. In addition, the compositions and methods can be particularly useful for sequencing from an array, where multiple sequences can be “read” simultaneously from multiple positions on the array since each nucleotide at each position can be identified based on its identifiable label. Example methods are described in US 2009/0088327; US 2010/0028885; and US 2009/0325172, each of which is incorporated herein by reference.

### Computer Systems

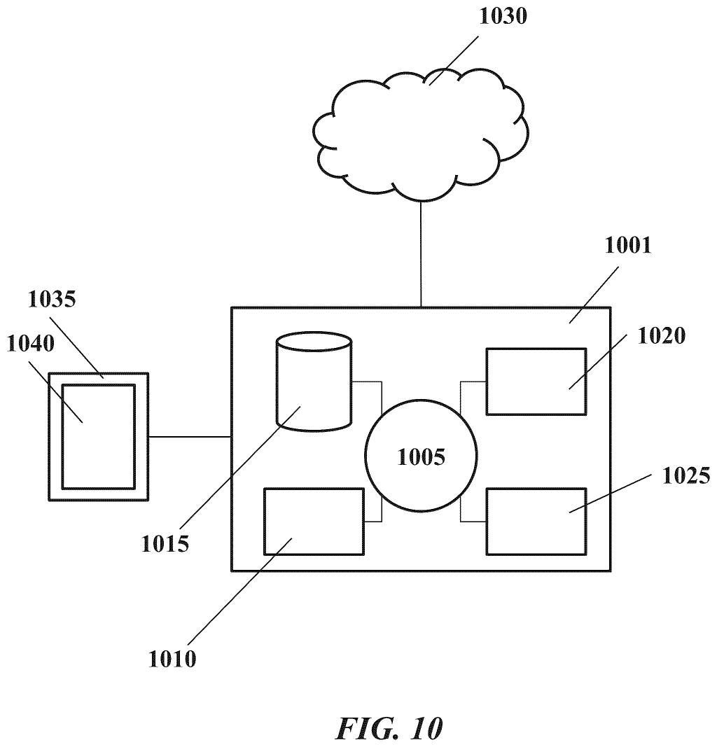

The present disclosure provides computer systems that are programmed to implement methods of the disclosure. FIG. 10 shows a computer system 1001 that is programmed or otherwise configured to analyze function and localization of therapeutic agents as described herein. The computer system 1001 can regulate various aspects of methods of the present disclosure. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the computer system 1001 via the network 1130.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software.

During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, assay conditions and protocols. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, be executed so as to detect a nucleic acid sequence utilizing methods and systems disclosed in the present disclosure. Optionally, the algorithms may be executed so as to control or effect operation of a component (e.g., light source, detector, reagent flow, etc) of the systems described herein to effect detection of a nucleic acid sequence.

### EXAMPLES

### Example 1—Pharmacodynamics (PD) Assays for Therapeutic Modalities

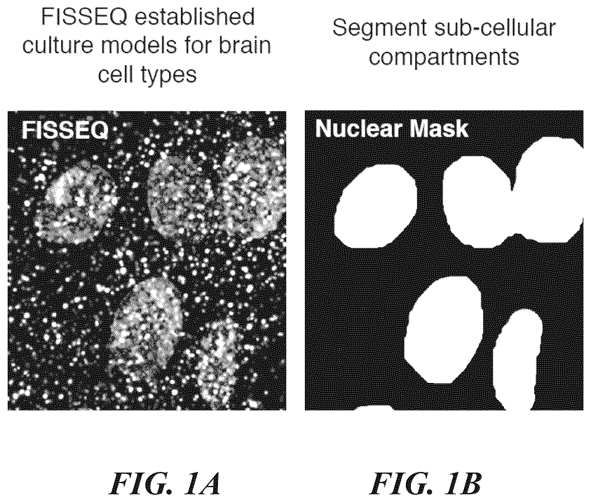

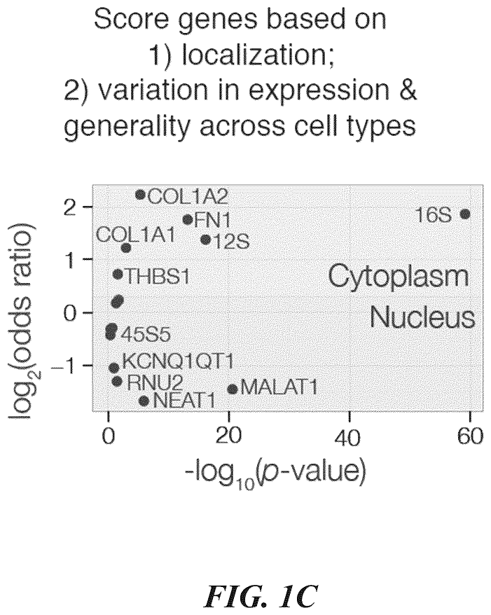

Minimal targeted FISSEQ PD knock-down assay of RNA interference (RNAi) and anti-sense modalities can be developed to detect PD of the anti-sense drug of interest in cells or tissue. FIG. 1A shows an example image of FISSEQ established culture models for brain cell types. Expression levels of the genes targeted directly or indirectly by anti-sense oligonucleotides (ASOs) were detected in the brain cells by FISSEQ. For example, the mRNAs of the target genes were detected. FIG. 1B shows the nuclei of the brain cells shown in FIG. 1A. The target genes were analyzed based on their sub-cellular localizations (e.g., cytoplasm or nucleus) and variation in expression in expression and generality across different cell types. FIG. 1C shows the genes affected by the ASOs, their sub-cellular localizations and the variations in expression levels.

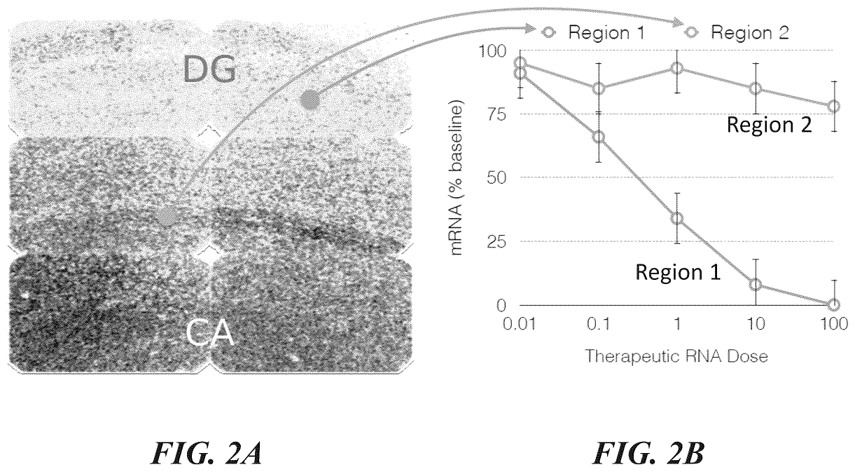

Minimal targeted FISSEQ PD knock-down assay can be developed to screen physical-chemical and receptor-mediated delivery mechanisms of RNAi and anti-sense modalities. FIG. 2A shows an example image of using minimal targeted FISSEQ knock-down assay to screen physical-chemical and receptor-mediated delivery mechanisms of RNAi and anti-sense modalities in intracerebroventricular (ICV) tissue model. The mRNA levels of a target gene in two different regions, cornu ammonis (CA) region and dentate gyrus (DG) region, were measured. FIG. 2B shows an example experimental data showing target mRNA level in response to therapeutic RNA dose in two different regions.

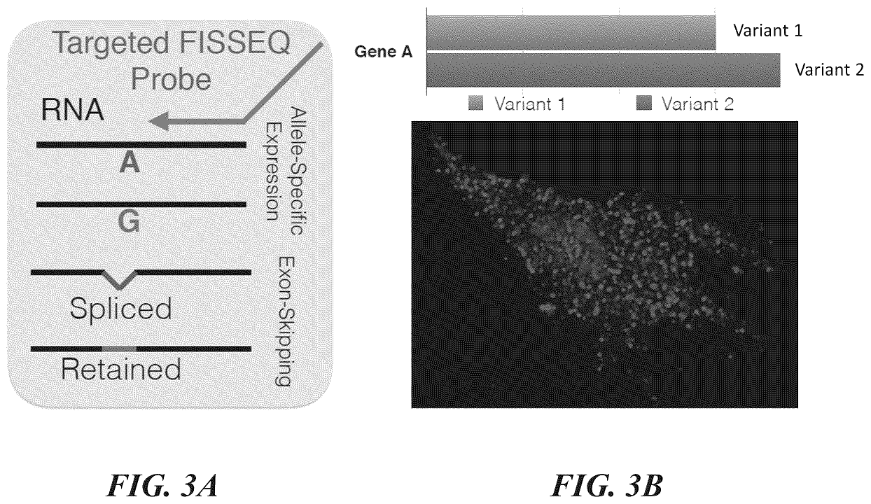

Validated culture models can be used to develop PD assay for allele-specific expression and exon-skipping. FIG. 3A shows examples of targeted FISSEQ used for allele-specific expression and exon-skipping of different variants of target genes. In allele-specific expression detection, a probe targeting the region adjacent to the position having different variants (e.g., A vs. G in FIG. 3A) on the RNA can be designed and the different variants can be detected by sequencing. In exon-skipping detection, a probe targeting the region adjacent to the exon to be skipped or retained can be designed and the presence or absence of the exon can be detected by sequencing. FIG. 3B shows an experimental image showing spatial information and expression level of two different variants (variant 1 and variant 2) of a target gene. The two different variants were detected by different fluorescent signals (e.g., fluorescent colors) as represented by different intensities shown in the image.

### Example 2-Atlas of Brain Cellular Organization

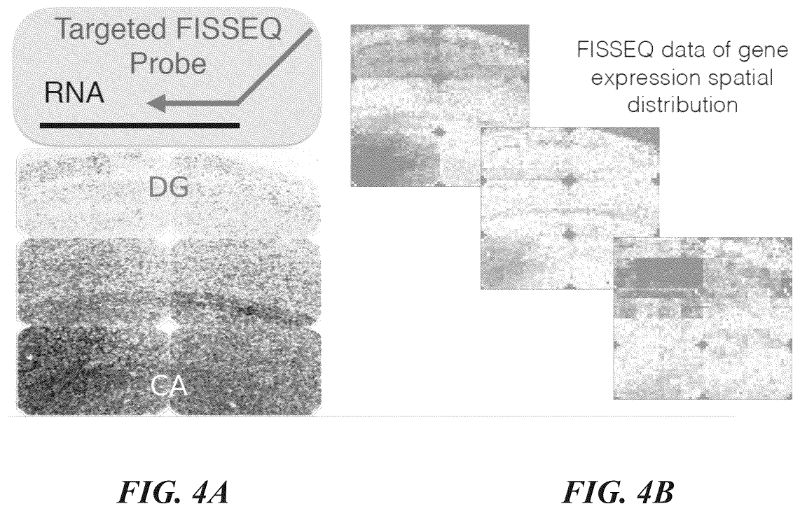

Targeted FISSEQ can be used to detect genes of interest to identify cell types. FIG. 4A shows an example design of targeted FISSEQ probe targeting an RNA of a gene of interest in a brain sample. The spatial information and expression level the gene of interest can be detected. FIG. 4B shows example FISSEQ data of gene expression spatial distribution. Using the assays shown in this example, gene expression spatial distribution of each gene of interest within a cell type can be detected, and spatial atlas of cell-type gene expression signatures can be developed. Moreover, whole transcriptome FISSEQ can be used for de novo discovery of gene expression signatures. In whole transcriptome FISSEQ, all mRNAs instead of selected mRNAs within a cell type can be detected. For example, all mRNAs can be targeted by a probe having a poly-deoxythymidine sequence. In addition, iterative data-driven curation of targeted FISSEQ library can be carried out for gene expression signatures based on whole-transcriptome profiling to achieve finer spatial resolution and to distinguish between subtle differences in cell type. Gene expression spatial distribution can also be used to detect, for example, immune response, inflammation, distribution of cell-membrane proteins for targeted delivery.

### Example 3-Co-Localization of Therapeutic Agents with Sub-Cellular Compartments

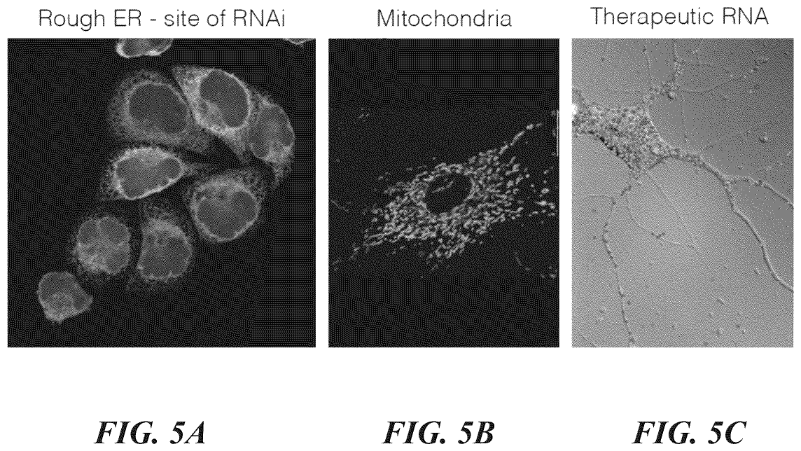

FISSEQ can be used to detect co-localization of therapeutic agent (e.g., small therapeutic RNAs) with sub-cellular anatomical features such as mitochondria, rough ER, P-bodies, and other relevant cellular compartments for therapeutic modalities and/or toxicity. FIG. 5A shows an example image of co-localization of RNAi and rough ER. FIG. 4B shows an example image of mitochondria within a cell. FIG. 5C shows an example image of therapeutic RNAs detected using FISSEQ within a cell.

### Example 4—Spatial In Situ Panomic Sequencing for Small RNA Localization and Transcriptional Effects

The example provides an assay that co-localizes an anti-sense oligonucleotide (ASO) and its gene target's transcriptional activation/repression at the single cell level. The assay can work in both tissue and cell culture. Selective detection of a therapeutic ASO versus a control ASO (F=255,589, p<0.001) and MALAT1 knockdown in the same assay (F=99,577, p<0.001) were demonstrated in this example. In addition, knockdown effect detection was validated with qPCR and gene detection was validated against bulk RNA sequencing (r=0.82, p<0.4). Such methods can allow screening of therapeutic candidate molecules with unprecedented fidelity and discrimination of their global and local pharmacokinetics (PK) and pharmacodynamics (PD).

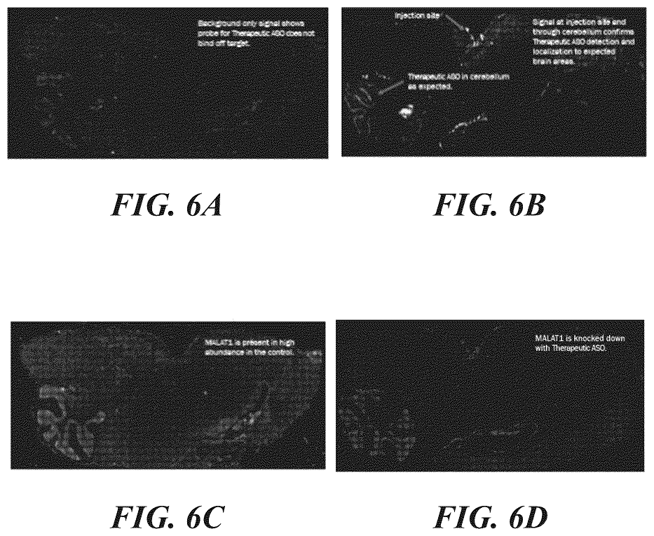

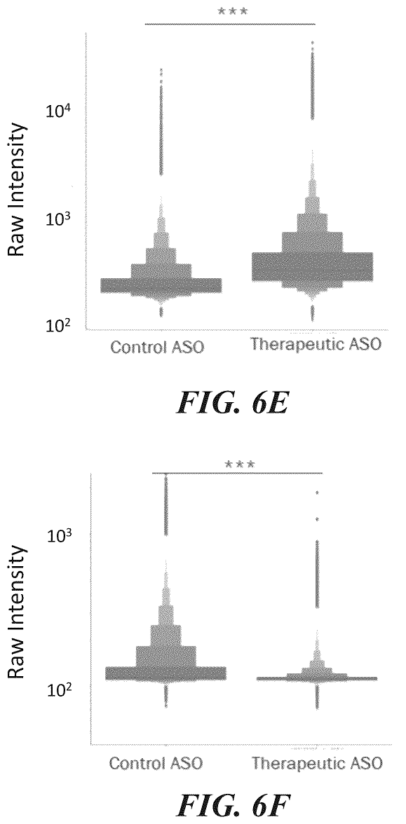

As illustrated in FIGS. 6A-F, the data show in situ anti-sense oligonucleotide (ASO) and knockdown detection in mouse brain: oligo FISSEQ designed to detect therapeutic ASO is selective against control ASO, which is in the background noise (ANOVA one-way F=255,589, p<0.001). FIG. 6A shows background signal of the mouse brain sample treated with control ASO. The probe targeting a therapeutic ASO does not bind to the control ASO. FIG. 6B shows that the therapeutic ASO, as detected by the probe targeting the therapeutic ASO, localized at the injection site (arrow on the top) and through cerebellum, and also shows high concentrations in the cerebellum as expected (arrow on the bottom). In addition, FIG. 6C shows high abundance of MALAT1 expression in the mouse brain sample treated with the control ASO and FIG. 6D shows that MALAT1 is knocked down in the mouse brain sample treated with the therapeutic ASO. FIG. 6E shows the raw intensity quantified from images in FIG. 6A and FIG. 6B. FIG. 6F shows raw intensity quantified from images in FIG. 6C and FIG. 6D. The assay validates MALAT1 is significantly knocked down by therapeutic vs inactive control (ANOVA one-way F=99,577, p<0.001).

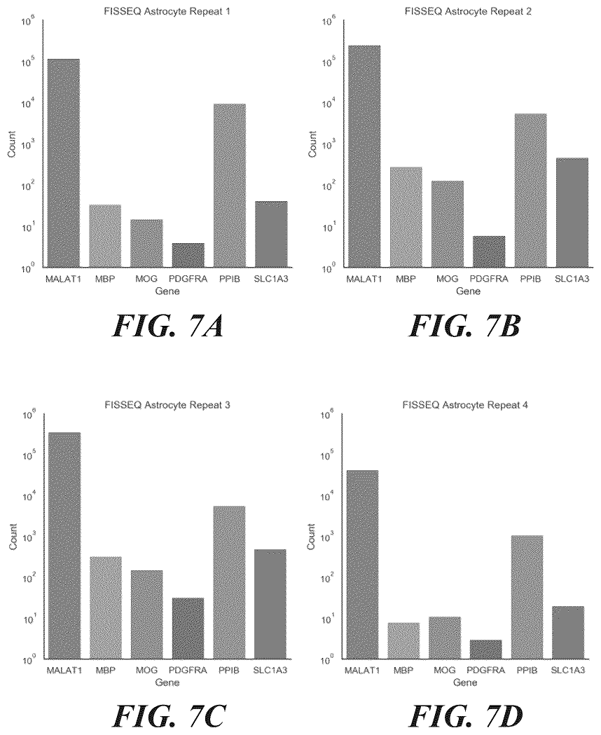

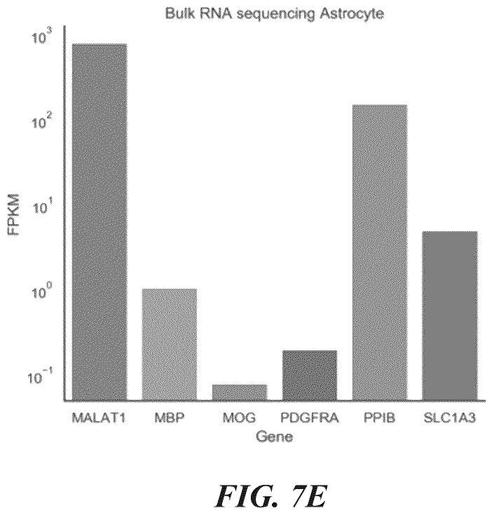

FIGS. 7A-D show four repeats of gene expression profile in astrocyte detected by FISSEQ. The count indicates the count number of unique barcodes detected, which was used to indicate the transcripts of each gene. FIG. 7E shows gene expression profile in astrocyte detected by bulk RNA sequencing. FPKM stands for fragments per kilobase of transcript per million mapped reads. As illustrated in FIGS. 7A-E, FISSEQ gene expression correlated with bulk RNA sequencing: bulk NGS sequencing (FIG. 7E) of astrocyte cell culture agrees with four replicates of FISSEQ (FIGS. 7A-D) of same cell culture (Spearman Sign Rank r=0.82, p<0.4). The genes tested in this example include MALAT1, MBP, MOG, PDGFRA, PPIB and SLC1A3.

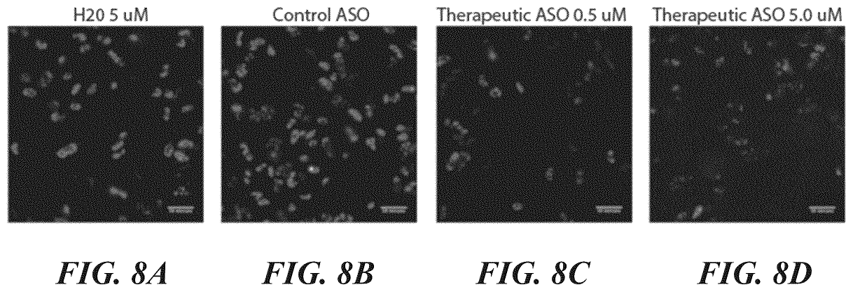

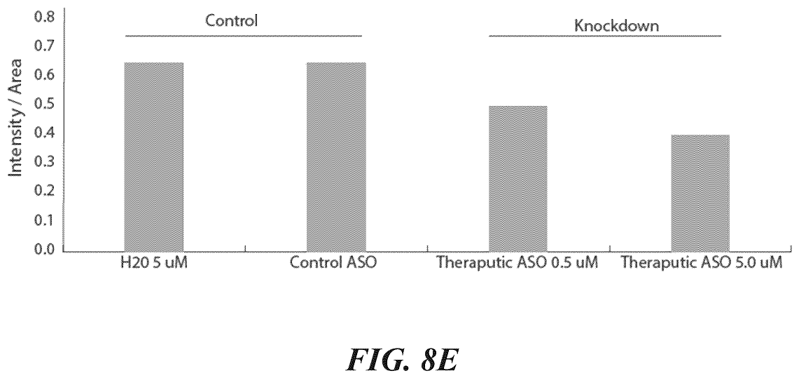

As illustrated in FIGS. 8A-E, the data show ASO knockdown of MALAT1 and localization in astrocyte cell culture dose response: dose response in cell culture of H₂O (FIG. 8A), control ASO (FIG. 8B), and therapeutic ASO at 0.5 μM (FIG. 8C) or 5 μM (FIG. 8D) to knockdown MALAT1. FIG. 8E shows the quantified intensities of signals shown in FIGS. 8A-D. The data show that intensity decreased as the concentration of the therapeutic ASO increased, indicating higher level of knockdown at higher concentration of therapeutic ASO.

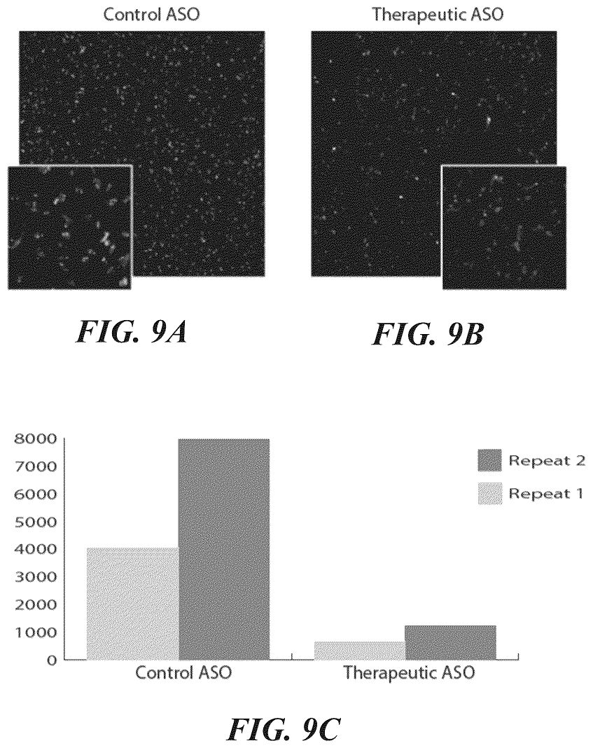

FIG. 9A shows MALAT1 detection in cell culture treated with control ASO. FIG. 9B shows MALAT1 detection in cell culture treated with therapeutic ASO. FIG. 9C shows a bar graph of qPCR data of the expression level of MALAT1. As illustrated in FIGS. 9A-C, the data show ASO knockdown in astrocyte cell culture correlated with qPCR: FISSEQ detected a ˜7-fold reduction in MALAT1 from therapeutic ASO 5 at μM compared to control ASO (two repeats exact Poisson Test p<0.01). Reduction corresponds qPCR7-fold reduction in same cell culture batch.

The assay described in this example can be used to co-localize small RNAs with their transcriptional effects in situ, and in the same sample. Using spatial panomic sequencing, the Pharmacokinetics (PK), Pharmacodynamics (PD), pathway analysis, and off target effects of these therapeutics can be investigated simultaneously, which can meaningfully expand the possible research and clinical experiments possible. Spatial panomic sequencing may have broad applications in precision health. In addition to RNAi therapeutics; for example, immuno-oncology may be a powerful means of treating tumors, however, the complex spatial density of ‘immunogenic’ cancer subtypes, stroma cells, and vasculature creates islands of effective and ineffective T-cells. NGS bulk sequencing mixes these subtypes and cannot distinguish the genetic, transcriptional, and vasculature innervation that predict treatment effectiveness. In contrast, sub-cellular spatial sequencing can create transcriptional, genetic, proteomic, and vasculature maps of a specific tumor environment to delineate the islands' subtypes and identify combinations of effective therapeutics.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-42. (canceled)

43. A method for identifying a target molecule and an anti-sense nucleic acid sequence, comprising:
(a) providing a biological sample having or suspected of having said target molecule and said anti-sense nucleic acid sequence;
(b) detecting a first set of signals and a second set of signals from said biological sample, wherein said first set of signals identifies a position of said anti-sense nucleic acid sequence in said biological sample, wherein said second set of signals identifies a change in a level of said target molecule relative to a reference, and wherein said first set of signals is detected with the aid of sequencing; and
(c) using said first set of signals and said second set of signals to provide an output indicative of said position of said anti-sense nucleic acid sequence and said target molecule in said biological sample.

44. The method of claim 43, further comprising, prior to (a), contacting said biological sample with a solution having said anti-sense nucleic acid sequence.

45. The method of claim 43, wherein said target molecule is a target nucleic acid molecule.

46. The method of claim 45, wherein (b) comprises identifying a target sequence of said target nucleic acid molecule.

47. The method of claim 43, wherein said target molecule is a target polypeptide or target protein.

48. The method of claim 43, wherein said biological sample is a cell, a cell derivative, or a tissue.

49. The method of claim 43, wherein said output is an image or a video.

50. The method of claim 43, wherein said anti-sense nucleic acid sequence is of a ribonucleic acid, a phosphorothioate deoxyribonucleic acid, a locked nucleic acid, a 2′-O-methocy-ethyl ribonucleic acid, a 2′-O-methyl ribonucleic acid, or a 2′-fluoro deoxyribonucleic acid.

51. The method of claim 43, wherein said anti-sense nucleic acid sequence is of an anti-sense nucleic acid molecule from 15 to 60 nucleotides in length.

52. The method of claim 43, wherein, in (b), said detecting is performed with a detection probe.

53. The method of claim 52, wherein said detection probe comprises a nucleic acid.

54. The method of claim 53, wherein (b) further comprises hybridizing said detection probe to said anti-sense nucleic acid sequence.

55. The method of claim 43, wherein said sequencing comprises sequencing-by-synthesis, sequencing-by-ligation, or sequencing by hybridization.

56. A method for identifying a target ribonucleic acid (RNA) molecule and an anti-sense RNA sequence, comprising:
(a) providing a biological sample having or suspected of having said target RNA molecule and said anti-sense RNA sequence, wherein said anti-sense RNA sequence is anti-sense to a sequence of said target RNA molecule;
(b) detecting a first set of signals and a second set of signals from said biological sample, wherein said first set of signals identifies a position of said anti-sense RNA sequence in said biological sample, and wherein said second set of signals identifies a change in a level of said target RNA molecule relative to a reference, wherein said first set of signals or said second set of signals are detected with the aid of sequencing; and
(c) using said first set of signals and said second set of signals to provide an output indicative of said position of said anti-sense RNA sequence and said target RNA molecule in said biological sample.

57. The method of claim 56, further comprising, prior to (a), contacting said biological sample with a solution having said anti-sense RNA sequence.

58. The method of claim 56, wherein said biological sample is a cell, a cell derivative, or a tissue.

59. The method of claim 56, wherein, in (b), said detecting is performed with a detection probe.

60. The method of claim 59, wherein (b) further comprises hybridizing said detection probe to said anti-sense RNA sequence.

61. The method of claim 59, wherein (b) comprises hybridizing said detection probe to a target sequence of said target RNA molecule.

62. The method of claim 56, wherein said sequencing comprises sequencing-by-synthesis, sequencing-by-ligation, or sequencing-by-hybridization.
